(19) United States
(12) Patent Application Publication
(10) Pub. No.: US 2014/0302170 A1
(43) Pub. Date: Oct. 9, 2014

(54) COMPOSITIONS AND METHODS FOR PROMOTING SLEEP

(71) Applicant: Scientific Nutrition Products, Inc., Cambridge, MA (US)

(72) Inventors: Robert C. Jones, Belmont, MA (US); Caroline Apovian, Waban, MA (US); Bruce R. Bistrian, Ipswich, MA (US); Judy Phillips, Arlington, MA (US)

(73) Assignee: Scientific Nutrition Products, Inc., Cambridge, MA (US)

(21) Appl. No.: 14/247,758

(22) Filed: Apr. 8, 2014

Related U.S. Application Data
(60) Provisional application No. 61/809,620, filed on Apr. 8, 2013.

(51) Int. Cl. A61K 31/405; A61K 45/06; A23L 1/305; A61K 31/4045

(52) U.S. Cl. CPC A61K 31/405; A61K 31/4045; A61K 45/06; A23L 1/3051
USPC 424/641; 514/419; 424/682; 424/722

(57) ABSTRACT

Described herein are food formulations and methods, which promote sleep.

### CROSS-REFERENCE TO RELATED APPLICATIONS

This application claims priority to U.S. Provisional Application No. 61/809,620 filed Apr. 8, 2013. The entire contents of this application is hereby incorporated by reference.

### BACKGROUND

All living organisms have intrinsic biological clocks or circadian rhythms that determine the timing of basic physiological occurrences, including the sleep-wake cycle. These rhythms cycle roughly every 24 hours and it is essential for the living being to synchronize its internal cycle to the external environment (daylight and nighttime, etc.) for well-being and survival. The brain essentially requires changes in autonomic and electrical activity roughly every 24 hours to maintain homeostasis in many domains. These changes are required to allow learning and memory to function ideally as well as manage brain energy balance in order to restore and rejuvenate brain cells.

In some animal models and humans, sleep cycles differ between the sexes due to gonadal hormones. However, both sexes are dependent on sleep for normal cognitive function, mental health and immune status. Indeed, when sleep cycles are not in synchrony with external light and dark cycles for long periods of time, humans develop disturbances such as emotional distress and lability, gastrointestinal dysfunction, and impaired immune status. Sleep deprivation has been used as a torture, and many days of sleep deprivation can lead to death of the organism.

Various studies have defined a normal sleep range as 7-8 hours per night. In these studies, “short sleep” is defined as habitual sleep time of 6 hours or less (Grander M A et al., Pack Sleep Medicine Reviews 2012; 14: 239-247; Dew MA et al., Psychosom Med 2003; 65(1):63-73). These studies have correlated an increase in mortality risk for those who sleep both less than and longer than the defined normal range. A comprehensive and systematic review of the literature has indicated that “short sleepers” (commonly less than seven hours per night, often less than five hours per night) have a 12% greater risk of mortality and “long sleepers” (commonly greater than eight or nine hours per night) a 30% greater risk of dying than those sleeping seven to eight hours per night. (Cappuccio, F P et al., Sleep 2008; 5:619-26).

“Short sleepers” are more likely to become chronically ill and to die prematurely, from coronary artery calcifications, cardiovascular risk predictors like hypertension, obesity, type 2 diabetes or impaired glucose control and atherogenic lipid profile. It is not clear why excessive sleep is also associated with an increase in mortality. However, associated factors such as depressive symptoms, low socioeconomic status, unemployment, low level of physical activity, undiagnosed health conditions, poor general health, and cancer-related fatigue may all contribute to mortality risk (Cappuccio, FP et al.)

The literature suggests that children 5 years of age require 12 hours per night, while adolescents require at least 9 hours per night. Elderly people, on the other hand, appear to need much less sleep. In a controlled study of 18 older (ages 60 to 76) and 35 younger (ages 18 to 32) healthy subjects, the older subjects slept for an average of 7.5 hours, while younger subjects slept an average of 9 hours (Klerman, E K and D Dijk Curr. Biol. 2008; 5:619-26).

It is estimated that sleep time has decreased in the general population in recent times by 2-3 hours per night (Webb W B et al., Bull Psychon Soc. 1975; 6:47-8). Insomnia is reported by approximately 50% of older adults who are generally dissatisfied with the quality of their sleep. In the US, the Center for Disease Control states, “Nationwide, an estimated 50 to 70 million people suffer from chronic sleep loss and sleep disorders.” (www.CDC.gov) According to data from the National Health Interview Survey, nearly 30% of adults reported an average of 6 hours or less of sleep per day in 2005-2007. In 2009, only 31% of high school students reported getting at least 8 hours of sleep on an average school night (www.CDC.gov). The CDC has called insomnia a public health epidemic.

The US leads the world in cases of insomnia (followed by Germany and England) and sleep problems add an estimated $15.9 billion to US national health care costs. Less sleep time is also documented in US children and adolescents (Lytle, LA et al, Obesity 2011: 19(2):324-31. Worldwide, at least 30% of adults and 70% of adolescents are not getting enough sleep.

Many professions necessitate shift work, and the Nurses' Health Study has documented that nurses who have done shift work for over 20 years have a 60% increase in the risk of type 2 diabetes.

Every year, 1.5 billion people in the United States, en masse, shorten sleep by one hour in the spring as we shift to Daylight Savings Time. Interestingly, in the week after that shift, there is a 16-17% increase in motor vehicle accidents and fatal motor vehicle accidents which are alcohol related. There is also a 5% increase in myocardial infarction that following week. This phenomenon reverses in the fall when the US gains one hour of sleep, and the week following there is a 5% decrease in myocardial infarction incidence. This alteration in events following a one hour difference in sleep time illustrates how sensitive the body is to sleep alteration.

Given that children sleep on average two hours less than was the norm decades ago, it also suggests that children are at high risk for learning and memory issues, as well as early disease, due to lack of sleep.

This decrease in sleep time has also been associated with an increase in obesity as measured by body mass index (BMI) (Patel, S R Obesity Reviews 2009:10 (Suppl. 2): 61-68). The mechanisms by which sleep duration is linked to BMI or weight gain have yet to be elucidated. However, current studies are focused on the role of several hormones, such as leptin, ghrelin, peptide YY (PYY), cortisol and others, which are generated by the gut and adipose tissue. They have been found to be significantly different in those who sleep 7-8 hours per night, compared to those who sleep 6 hours or less per night.

In addition to hypertension, type 2 diabetes, and metabolic syndrome (any of which could lead to mortality), deficits in executive functioning, learning, and memory have also been linked with short sleep. (Grandner, M et al., Pack Sleep Medicine Reviews 2012; 14: 239-247). Impaired executive functioning can manifest as impaired decision making and risky behavior. Psychological impairment from sleep deprivation can manifest as depression, anxiety, and mood dysregulation. (Kilgore W D et al., J. Sleep Res Mar 2006 ; 15(1) :7-13.

Different types of studies have consistently shown a relationship between sleep and body mass index (BMI). However, as stated initially, this relationship differs between the sexes. Increased BMI is associated with decreased sleep duration in men, but there is a U-shaped relationship in women, such that high BMI is seen in women with short sleep and also with prolonged sleep duration (Littman, A J et al., Int Jobes Mar 2007; 31(3):466-75; Patel, S R Obesity Reviews 2009:10 (Suppl. 2): 61-68).

In these studies, the lowest BMI was reported at around 7.7 hours sleep duration per night. Sleep duration less than 7 hours has been shown to be associated with increased risk of weight gain in longitudinal studies of self-reported sleep durations. In fact, a study that collected data over 13 years reported that every extra hour increase in sleep duration is associated with a 50% reduction in risk of obesity (Hasler, G et al., Sleep June 2004; 27(4) 661-6. This report must be tempered by the U-shaped relationship of sleep and BMI, which may be confounded by depression, for example, in those with high BMI and long sleep duration.

Adults who sleep 5-7 hours per night or less are 30-80% more likely to develop Type 2 diabetes, cardiovascular disease, hypertension and premature death as compared to those who sleep 8 hours or more. Chronic sleep deprivation increases appetite, levels of pro-inflammatory cytokines in the blood, blood pressure, evening cortisol and insulin and blood glucose levels (McKeon-Cowdin R, BMC Com Alt Med 2011; Nov 8;11:109; Hotamisligil, GS Nature (2006) 444: 860-7).

The increase in cortisol that occurs after short sleep can cause an increase in heart rate and blood pressure, as cortisol is a stress hormone. In addition, elevations in cortisol have been shown to increase the risk of weight gain, specifically abdominal weight gain, which is more highly correlated with disease such as type 2 diabetes, and cardiovascular disease.

Among children and adolescents, less sleep is correlated with academic and behavioral problems, in addition to elevated blood pressure and early type 2 diabetes. Studies in younger populations have shown that sleep deprivation is both a neurobiological and physiological stressor that can cause metabolic derangements.

Inadequate sleep time has been found to be a risk factor for adolescent obesity (Lytle, L A et al., Obesity 2011: 19:324-31). BMI is also inversely associated with sleep duration in adults. Multiple studies have found this association, and a recent meta-analysis has corroborated this relationship (Patel, S R and F B Hu Obesity 2008: 3: 643-53). More importantly, the mechanism of action can be extrapolated (but not proven as yet) to be a combination of increased hunger, increased fatigue, altered thermoregulation, and perhaps even more opportunity to eat due to less sleep time. Studies have shown that hormonal changes—namely increases in ghrelin and decreases in leptin—in those who sleep less have been associated with increased hunger based on visual analogue scales. It has also been shown that less sleep time causes a decrease in core temperature, which, if chronically associated with less sleep time, can be another mechanism leading to weight gain (due to decreased resting energy expenditure). The hypothesis that less sleep time produces more opportunity to eat is intriguing, based on the habits of many obese persons who eat in the evening up until bed time. Food is generally restricted in the habits of these patients during the day, leading to a vicious cycle which can perpetuate later bedtime.

“Short sleep” has been associated with metabolic syndrome in women, but this association may have been confounded by the sleep disturbances that are often seen in the menopause (Choi, J K et al., J. Exp. Med. 2011 225(3):187-93). Menopause disrupts the sleep cycle and hormonal replacement can normalize this derangement. Menopause has also been associated with higher prevalence of depression and weight gain. The interrelationship among menopause, weight gain and sleep is therefore complex and associations can be misleading. It may be, in fact, that menopause leads to disruption in sleep which then leads to weight gain through multiple mechanisms including depression and mood disorder. There is also recent evidence that “short sleep” leads to increased levels of ghrelin and decreased levels of PYY, leading to increased hunger and decreased satiety (Taheri S et al., PloS Med 2004; 3: e62). It could be that several factors occurring at similar time points in the lifecycle of a woman (here, menopause) tend to promote weight gain, which makes it difficult to study one factor without controlling for the other variables.

There is a need for new compositions and methods to promote sleep.

### SUMMARY

Featured herein are food formulations, which when ingested by an individual (e.g. a mammal, such as a human) induces and/or maintains sleep in that individual. An appropriate formulation may provide an appropriate amount of melatonin, an appropriate amount of L-tryptophan and/or an appropriate amount of a high glycemic index carbohydrate. An appropriate formulation may have a total carbohydrate to protein ratio, which is less than or equal to about 10:1. An appropriate formulation may have a relatively high concentration of tryptophan as compared to other large neutral amino acids (i.e. leucine, isoleucine, valine, tyrosine and phenylalanine). An appropriate formulation may be in any physical state, e.g. a liquid, gas or solid.

Melatonin may be provided by a food selected from the group consisting of: cherries, olive oil, tomatoes, grapes, walnuts, grains and rices. Appropriate cherries may be tart, (i.e. have a relatively low sugar content). Examples include Montmorency and Jerte Valley cherries. The total amount of melatonin in the formulation may be at least about 0.3 mgs. and less than about 10 mgs. For example, the amount of melatonin may be in the range of about 0.3 to about 1.0 mgs.

L-tryptophan may be provided in a food selected from the group consisting of: meats, seafoods, milks, cheeses, whey protein (including its various constituent peptides), vegetables, fruits (e.g. bananas) legumes (e.g. soy), whole grain foods, rices or nuts. In addition, proteins or peptides, which are rich in tryprophan, and preferably limited in leucine, may also be incorporated into the food formulation. The total amount of L-tryptophan in the formulation may be at least about 3 grams. For example, the amount of L-tryptophan in the formulation may be in the range of about 3 to about 5 grams.

High glycemic index carbohydrates may be provided, for example, in a food selected from the group consisting of: glucose, maltose, maltodextrin, potato, pretzel, a fruit, a grain, parsnip, white bread (wheat endosperm only), white rice (rice endosperm only), a corn flake or an extruded breakfast cereal.

An appropriate formulation should not include foods which contain an ingredient that negatively impacts or disrupts sleep, such as caffeine, alcohol, certain spices or high calorie, high sugar or fatty foods.

Sleep promoting food formulations may optionally include foods, which contain additional sleep promoting ingredients, such as zinc (e.g. pumpkin seeds, dark chocolate, garlic and sesame seeds); magnesium (e.g. bran (rice, wheat or oat); oatmeal; dried herbs (coriander, chives, spearmint, dill, sage, basil and savory); squash, pumpkin or watermelon seeds; dark chocolate; flax, sesame seeds or sesame butter; brazil nuts; sunflower seeds; almonds; cashews; molasses; or dry roasted soybeans); taurine (e.g. fish, meat, breast milk, sea algae and sea plants); 5-hydroxytryptophan (e.g. milk and dairy, meat, nuts, seeds, fish, fruit, vegetables, dark chocolate); potassium (e.g. bananas, potatoes, prune juice, plums, oranges, orange juice, tomatoes, spinach, sunflower seeds and almonds) and L-theanine (e.g. chamomile, green or black tea or bay bolete mushrooms).

Also featured are methods for inducing and/or promoting sleep in an individual comprising the step of: having the individual consume an appropriate amount of a formulation at an appropriate period of time in advance of when the individual would like to fall asleep. For example, the sleep promoting formulation may be taken at least 15, 30, 45, 60 or 75 minutes before an individual wishes to fall asleep.

In addition to consuming an appropriate formulation, an individual may undertake another approach towards attaining and/or maintaining sleep, such as: 1) ensuring that the area where the sleep is to occur is sufficiently dark; 2) reducing stress by use of techniques such as biofeedback, other meditative or relaxation techniques or massage; 3) avoiding strenuous exercise immediately prior to sleeping and/or 4) avoiding consumption of foods that contain sleep disturbing ingredients immediately prior to sleeping.

Food formulations described herein have been clinically demonstrated to promote sleep; are pleasant tasting and are relatively low in calories.

Further features and advantages will become apparent from the following Detailed Description and Claims.

### DETAILED DESCRIPTION

### Food Formulations

Described herein are food formulations, which when ingested by an individual (e.g. a human or other mammal) promotes and/or maintains sleep in that individual. As used herein, food refers to any substance, which may be consumed by a subject to provide nutritional support for the body. A food may be in any state, including a solid, a liquid or a gas. For example, a fruit may be in the solid form in which it exists in nature or as a fruit juice or extract. In addition, a food formulation may be comprised of multiple foods or components of foods, such as constituent proteins, peptides, carbohydrates, lipids etc.

As described herein, appropriate food formulations contain an appropriate amount of melatonin, L-tryptophan and a high glycemic index carbohydrate. When ingested by an individual, the formulation induces and maintains sleep for an appropriate period of time (e.g. 6, 7, 8, 9, 10, 11 or 12 hours). The ratio of total carbohydrate to total protein in a food formulation may be less than or equal to about 10:1.

Melatonin, also known as N-acetyl-5-methoxy-tryptamine is a naturally occurring compound found in animals, plants and microbes. Melatonin can be present in a variety of foods, including cherries (especially tart cherries, such as Montmorency or Jerte Valley cherries), olive oil, tomatoes, grapes, walnuts, grains and rices. The amount of melatonin in an appropriate food formulation may be at least about 0.3 mgs and less than about 10 mgs. For example, the amount of melatonin can be in the range of about 0.3 to about 1.0 mgs.

When the melatonin is provided by tart cherries, it may be in the form of a juice concentrate. For example, the melatonin may be provided in a tart cherry juice concentrate. For example, less than about 50 mls. (e.g. 30 mls).

L-tryptophan is one of 22 standard, naturally occurring amino acids and is considered an essential amino acid in the human diet. L-tryptophan may be contained in the following foods: meats, seafoods, fruits (bananas), milks, cheeses, whey protein (including its various constituent peptides), vegetables, fruits (e.g. bananas) legumes (e.g. soy), whole grain foods, rices or nuts. In addition, proteins or peptides, which are rich in tryprophan, and preferably limited in leucine, may also be incorporated into the food formulation. The amount of L-tryptophan in an appropriate food formulation may be at least about 3 grams. The amount may be less than about 10, 9, 8, 7 or 6 grams.

“High glycemic index carbohydrate” refers to a carbohydrate, which when ingested by a subject breaks down relatively quickly during digestion and releases glucose rapidly into the subject's blood stream and elicits a prompt insulin response. Insulin then serves to reduce the branched chain amino acid content of the plasma and alters the ratio of tryptophan/branched chain amino acids that, by using the same transport system into the brain, increases brain transport of tryptophan. This effect the serves to extend the increased availability of tryptophan to support melatonin production for up to 5 hours and supplements the initial effects of pre-formed melatonin. High glycemic index is typically defined as 70 and above. Examples of foods having high glycemic index carbohydrates include glucose (GI=100), maltose, maltodextrin, potatoes, pretzels, parsnips, white breads (wheat endosperm only), white rices (rice endosperm only), corn flakes and extruded breakfast cereals.

An appropriate formulation should not include an ingredient that prevents sleep. Examples include: caffeine, alcohol, certain spices or foods, which contain a lot of calories, fat or sugar.

Appropriate formulations may optionally include additional sleep promoting ingredients, such as zinc (present, for example, in pumpkin seeds, dark chocolate, garlic and sesame seeds); magnesium (present, for example in brans (rice, wheat or oat); dried herbs (coriander, chives, spearmint, dill, sage, basil and savory); squash, pumpkin or watermelon seeds; dark chocolate; flax, sesame seeds or sesame butter; brazil nuts; sunflower seeds; almonds; cashews; molasses; or dry roasted soybeans (edamame); taurine (e.g. fish, meat, breast milk, sea algae and sea plants); 5-hydroxytryptophan (e.g. milk and dairy, meat, nuts, seeds, fish, fruit, vegetables, dark chocolate); potassium (e.g. bananas, potatoes, prune juice, plums, oranges, orange juice, tomatoes, spinach, sunflower seeds and almonds) and L-theanine (e.g. chamomile, green or black tea or bay bolete mushrooms). taurine (e.g. fish, meat, breast milk, sea algae and sea plants); 5-hydroxytryptophan (e.g. milk and dairy, meat, nuts, seeds, fish, fruit, vegetables, dark chocolate); potassium (e.g. bananas, potatoes, prune juice, plums, oranges, orange juice, tomatoes, spinach, sunflower seeds and almonds) and L-theanine (e.g. chamomile, green or black tea or bay bolete mushrooms).

### Methods

Also featured are sleep promotion methods. A sleep promotion method may comprise the step of consuming an appropriate amount of a sleep promoting food formulation at an appropriate time in advance of going to bed. For example, the sleep promoting formulation may be taken at least 15, 30, 35, 60 or 75 minutes before an individual wishes to fall asleep.

In addition to consuming an appropriate formulation, an individual may take one or more other steps aimed at promoting or maintaining sleep, such as ensuring that the area where the sleep is to occur is sufficiently dark; stress reduction; avoiding strenuous exercise or consuming foods with sleep disturbing ingredients immediately prior to such time as an individual wishes to go to sleep.

The invention, now being generally described, will be more readily understood by reference to the following example, which is included merely for purposes of illustration of certain aspects and embodiments and is not intended to limit the invention.

### The Example: Method for Making a Sleep Promoting Formulation

| Ingredient | % w/w | g/dose |
|---|---|---|
| Red tart frozen Cherry Juice Conc. (68 brix) | 26.000 | 19.500 |
| Tart Cherry Juice powder (CherryPure) 80 mesh | 1.893 | 1.420 |
| Pepform (Tryptophan source) | 6.400 | 4.800 |
| Xanthan gum | 0.200 | 0.150 |
| Reb-A 99% 903064 | 0.067 | 0.050 |
| NAT Symlife Sweet Flavor 312019 | 0.500 | 0.375 |
| NAT Cherry Pomegranate flavor 167547 | 0.300 | 0.225 |
| Water | 64.640 | 48.480 |
| TOTAL | 100.000 | 75.000 |

1. Combine Cherry Juice Concentrate, cherry juice powder, xanthan gum and water. Mix with heat to 70-80° C.
2. Add Pepform powder and mix until uniform (visually red with no suspended particles).
3. Remove from heat.
4. Add Reb-A and flavors. Mix until uniform.

Although not wishing to be bound by theory, it would appear that the tart cherry juice provided in the formulation provides a ready source of melatonin, which promotes sleep in the initial 30 minutes after ingestion. The cherry juice also provides a source of high-glycemic index carbohydrates. Tryptophan is also provided in a special protein mixture. The combination facilitates the transport of tryptophan across the blood: brain barrier. Via a series of well-established pathways, the tryptophan converts to serotonin, which then converts to melatonin over several hours, thereby providing a second wave of melatonin, which promotes sleep several hours after falling asleep. The formulation helps one to fall asleep fairly rapidly and stay asleep for a sufficient period of time.

1. A sleep promoting food formulation comprising: an appropriate amount of melatonin, an appropriate amount of L-tryptophan and an appropriate amount of high glycemic index carbohydrates.

2. A formulation of claim 1, wherein the total carbohydrate to protein ratio of the formulation is less than or equal to about 10:1.

3. A formulation of claim 1, which has a larger concentration of tryptophan than other large neutral amino acids.

4. A formulation of claim 1, which is a liquid.

5. A formulation of claim 1, wherein the melatonin is provided by a food selected from the group consisting of: cherries, olive oil, tomatoes, grapes, walnuts, grains and rices.

6. A formulation of claim 1, wherein the L-tryptophan is provided in a food selected from the group consisting of: a meat, seafood, milk, cheese, whey protein, vegetable, fruit, legume, whole grain food, rice, nut, protein rich in tryptophan and peptide rich in tryptophan.

7. A formulation of claim 1, wherein the high glycemic index carbohydrate is provided in a food selected from the group consisting of: glucose, maltose, maltodextrin, potato, pretzel, a fruit, a grain, parsnip, a white bread, a white rice, a corn flake or extruded cereal.

8. A formulation of claim 1, which does not contain caffeine, alcohol or a high calorie, high sugar and/or fatty food.

9. A formulation of claim 1, which additionally comprises an ingredient selected from the group consisting of zinc, magnesium, taurine, 5-hydroxytryptophan, potassium and L-theanine

10. A formulation of claim 1 as described in the example.

11. A method for promoting sleep and/or maintaining a sleep state in an individual comprising the step of: having the individual consume an appropriate amount of a sleep promoting formulation of claim 1 at an appropriate period of time in advance of when the individual would like to fall asleep.

12. A method of claim 11, wherein the appropriate period of time is selected from the group consisting of 15, 30, 45, 60 or 75 minutes prior to when the individual would like to fall asleep.

13. A method of claim 11, wherein the individual also sleeps in the dark and/or has minimized stress, strenuous exercise or the consumption of sleep disturbing ingredients immediately prior to when the individual would like to fall asleep.
